SEQUENCE LISTING (63 sequences)

SEQ ID NO: 1 - protein, 4 aa, Unknown
  unsure of organism origin
DEVD

SEQ ID NO: 2 - protein, 4 aa, Unknown
  unsure of organism origin
YVAD

SEQ ID NO: 3 - protein, 6 aa, Unknown
  unsure or organism origin
HFRWXK

SEQ ID NO: 4 - protein, 7 aa, Unknown
  unsure of organism origin
HFRWXXK

SEQ ID NO: 5 - protein, 8 aa, Unknown
  unsure of organism origin
HFRWXXXK

SEQ ID NO: 6 - protein, 9 aa, Unknown
  unsure of organism origin
HFRWXXXXK

SEQ ID NO: 7 - protein, 10 aa, Unknown
  unsure of organism origin
HFRWXXXXXK

SEQ ID NO: 8 - protein, 7 aa, Unknown
  unsure of organism origin
HFRWXKP

SEQ ID NO: 9 - protein, 8 aa, Unknown
  unsure of organism origin
HFRWXXKP

SEQ ID NO: 10 - protein, 9 aa, Unknown
  unsure of organism origin
HFRWXXXKP

SEQ ID NO: 11 - protein, 10 aa, Unknown
  unsure of organism origin
HFRWXXXXKP

SEQ ID NO: 12 - protein, 11 aa, Unknown
  unsure of organism origin
HFRWXXXXXKP

SEQ ID NO: 13 - protein, 8 aa, Unknown
  unsure of organism origin
HFRWXKPW

SEQ ID NO: 14 - protein, 9 aa, Unknown
  unsure of organism origin
HFRWXXKPW

SEQ ID NO: 15 - protein, 10 aa, Unknown
  unsure of organism origin
HFRWXXXKPW

SEQ ID NO: 16 - protein, 11 aa, Unknown
  unsure of organism origin
HFRWXXXXKPW

SEQ ID NO: 17 - protein, 12 aa, Unknown
  unsure of organism origin
HFRWXXXXXKPW

SEQ ID NO: 18 - protein, 8 aa, Unknown
  unsure of organism origin
HFRWXKPV

SEQ ID NO: 19 - protein, 9 aa, Unknown
  unsure of organism origin
HFRWXXKPV

SEQ ID NO: 20 - protein, 10 aa, Unknown
  unsure of organism origin
HFRWXXXKPV

SEQ ID NO: 21 - protein, 11 aa, Unknown
  unsure of organism origin
HFRWXXXXKPV

SEQ ID NO: 22 - protein, 12 aa, Unknown
  unsure of organism origin
HFRWXXXXXKPV

SEQ ID NO: 23 - protein, 5 aa, Unknown
  unsure of organism origin
FRWXK

SEQ ID NO: 24 - protein, 6 aa, Unknown
  unsure of organism origin
FRWXXK

SEQ ID NO: 25 - protein, 7 aa, Unknown
  unsure of organism origin
FRWXXXK

SEQ ID NO: 26 - protein, 8 aa, Unknown
  unsure of organism origin
FRWXXXXK

SEQ ID NO: 27 - protein, 9 aa, Unknown
  unsure of organism origin
FRWXXXXXK

SEQ ID NO: 28 - protein, 6 aa, Unknown
  unsure of organism origin
FRWXKP

SEQ ID NO: 29 - protein, 7 aa, Unknown
  unsure of organism origin
FRWXXKP

SEQ ID NO: 30 - protein, 8 aa, Unknown
  unsure of organism origin
FRWXXXKP

SEQ ID NO: 31 - protein, 9 aa, Unknown
  unsure of organism origin
FRWXXXXKP

SEQ ID NO: 32 - protein, 10 aa, Unknown
  unsure of organism origin
FRWXXXXXKP

SEQ ID NO: 33 - protein, 7 aa, Unknown
  unsure of organism origin
FRWXKPT

SEQ ID NO: 34 - protein, 8 aa, Unknown
  unsure of organism origin
FRWXXKPT

SEQ ID NO: 35 - protein, 9 aa, Unknown
  unsure of organism origin
FRWXXXKPT

SEQ ID NO: 36 - protein, 10 aa, Unknown
  unsure of organism origin
FRWXXXXKPT

SEQ ID NO: 37 - protein, 11 aa, Unknown
  unsure of organism origin
FRWXXXXXKPT

SEQ ID NO: 38 - protein, 7 aa, Unknown
  unsure of organism origin
FRWXKPV

SEQ ID NO: 39 - protein, 8 aa, Unknown
  unsure of organism origin
FRWXXKPV

SEQ ID NO: 40 - protein, 9 aa, Unknown
  unsure of organism origin
FRWXXXKPV

SEQ ID NO: 41 - protein, 10 aa, Unknown
  unsure of organism origin
FRWXXXXKPV

SEQ ID NO: 42 - protein, 11 aa, Unknown
  unsure of organism origin
FRWXXXXXKPV

SEQ ID NO: 43 - protein, 5 aa, Unknown
  unsure of organism origin
HFRXK

SEQ ID NO: 44 - protein, 6 aa, Unknown
  unsure of organism origin
HFRXXK

SEQ ID NO: 45 - protein, 7 aa, Unknown
  unsure of organism origin
HFRXXXK

SEQ ID NO: 46 - protein, 8 aa, Unknown
  unsure of organism origin
HFRXXXXK

SEQ ID NO: 47 - protein, 9 aa, Unknown
  unsure of organism origin
HFRXXXXXK

SEQ ID NO: 48 - protein, 6 aa, Unknown
  unsure of organism origin
HFRXKP

SEQ ID NO: 49 - protein, 7 aa, Unknown
  unsure of organism origin
HFRXXKP

SEQ ID NO: 50 - protein, 8 aa, Unknown
  unsure of organism origin
HFRXXXKP

SEQ ID NO: 51 - protein, 9 aa, Unknown
  unsure of organism origin
HFRXXXXKP

SEQ ID NO: 52 - protein, 10 aa, Unknown
  unsure of organism origin
HFRXXXXXKP

SEQ ID NO: 53 - protein, 7 aa, Unknown
  unsure of organism origin
HFRXKPT

SEQ ID NO: 54 - protein, 8 aa, Unknown
  unsure of organism origin
HFRXXKPT

SEQ ID NO: 55 - protein, 9 aa, Unknown
  unsure of organism origin
HFRXXXKPT

SEQ ID NO: 56 - protein, 10 aa, Unknown
  unsure of organism origin
HFRXXXXKPT

SEQ ID NO: 57 - protein, 11 aa, Unknown
  unsure of organism origin
HFRXXXXXKPT

SEQ ID NO: 58 - protein, 7 aa, Unknown
  unsure of organism origin
HFRXKPV

SEQ ID NO: 59 - protein, 8 aa, Unknown
  unsure of organism origin
HFRXXKPV

SEQ ID NO: 60 - protein, 9 aa, Unknown
  unsure of organism origin
HFRXXXKPV

SEQ ID NO: 61 - protein, 10 aa, Unknown
  unsure of organism origin
HFRXXXXKPV

SEQ ID NO: 62 - protein, 11 aa, Unknown
  unsure of organism origin
HFRXXXXXKPV

SEQ ID NO: 63 - protein, 6 aa, Unknown
  unsure of organism origin
HFRKPD

(19) United States
(12) Patent Application Publication
(10) Pub. No.: US 2009/0232866 A1
(43) Pub. Date: Sep. 17, 2009

(54) Oligopeptides as coating material for medical products

(76) Inventors: Mariann Pavone-Gyongyosi, Brunn am Gebirge (AT); Helmut Dietmar Glogar, Wien (AT); Erika Hoffmann, Eschweiler (DE)

Correspondence Address: Amin & Turocy, LLP, 24th Floor, National City Center, 1900 East 9th Street, Cleveland, OH, 44114, US

(21) Appl. No.: 10/680,035

(22) Filed: Oct. 7, 2003

(51) Int. Cl. A61K 9/00; A61K 38/05; A61K 38/06; A61K 38/07; A61K 38/08; A61K 38/10; A61K 38/16; B05D 3/00; A61P 9/00

(52) U.S. Cl. 424/423; 514/19; 514/18; 514/17; 514/16; 514/15; 514/14; 514/13; 514/12; 427/2.1

(57) ABSTRACT

The present invention relates to a pharmaceutical composition comprising a caspase inhibitor and/or a compound of the general formula R-Lys-X, methods for coating medical products using said caspase inhibitors and/or said compounds of general formula R-Lys-X and medical products coated with said caspase inhibitors and/or said compounds of general formula R-Lys-X.

### FIELD OF THE INVENTION

The present invention relates to a pharmaceutical composition comprising a caspase inhibitor and/or a compound of the general formula R-Lys-X, methods for coating medical products using said caspase inhibitors and/or said compounds of general formula R-Lys-X and medical products coated with said caspase inhibitors and/or said compounds of general formula R-Lys-X.

### BACKGROUND OF THE INVENTION

In connection with coronary interventions and especially with the percutaneous transluminal coronary angioplasty (PTCA) it was demonstrated that this kind of non-surgical therapy is limited because of a restenosis rate of up to 35%. Several investigations show that the balloon angiography and also the stent implantation causes injuries and the tear of plaques and vascular walls, leading to neointimal hyperplasty and proliferation of smooth muscle cells.

Said smooth muscle cells generate an extracellular matrix in the newly formed intima. Furthermore, the injuries cause local inflammations and the migration of lymphocytes, macrophages and monocytes into the newly formed intima. This neointimal proliferation causes restenosis and methods are desired which reduce the risk of restenosis by controlling the proliferation and diminishing the inflammatory processes.

Object of the present invention is to provide compounds and pharmaceutical compositions for the reduction of restenosis, coating of medical products which reduce the risk of restenosis and methods for manufacturing said coated medical products.

The object is solved by the teaching of the independent claims. Further advantageous embodiments of the present invention are evident from the dependent claims, the description and the examples.

### DESCRIPTION OF THE INVENTION

The present invention relates to the use of caspase inhibitors and/or at least one compound of the general formula R-Lys-X for the preparation of a pharmaceutical composition, the use of said caspase inhibitor and/or said at least one compound of the general formula R-Lys-X or said pharmaceutical composition for coating surfaces of medical products, especially of stents. Furthermore, the present invention relates to medical products coated according to the invention coating method, especially to stents coated according to the inventive methods.

Caspases are widely conserved proteases considered to be essential effectors of apoptosis.

A wide range of caspase inhibitors are known of which peptidic caspase inhibitors such as benzyloxycarbonyl-Val-Ala-Asp-fluoromethyl ketone or Ile-Glu-Thr-Asp-fluoromethyl ketone are the most popular examples. Preferable are caspase inhibitors in the form of free or protected peptides consisting of two, three, four or five amino acids.

Caspase inhibitors consisting of only one amino acids are nevertheless also useful to be applied to the present invention. Examples of said inhibitors comprise for instance t-butoxycarbonyl-Asp(OCH₃)—CH₂F, boc-aspartyl(OMe)-fluoromethylketone (BAF) and BOC-Asp-FMK (BD).

Examples for dipeptides as caspase inhibitors are BD-fmk and Z-FA-fmk.

Examples for tripeptides as caspase inhibitors are z-VAD, z-Val-Ala-Asp-fluoromethylketone (z-VAD-fmk), IAP, benzyloxycarbonyl-Val-Ala-Asp(OCH₃)—CH₂-fluoromethyl ketone, benzyloxycarbonyl-Ile-Glu(OCH₃)-Thr-Asp(OCH₃)—CH₂-fluoromethyl ketone and Z-AAD-fmk.

Examples for tetrapeptides as caspase inhibitors are DEVD, Ac-DEVD-CHO, Z-Asp-CH₂-DCB, acetyl-Asp-Glu-Val-Asp-fluoromethyl-ketone (Ac-DEVD-FMK), YVAD, acetyl-Tyr-Val-Ala-Asp-chloromethyl-ketone (Ac-YVAD-CMK), z-DEVD-fmk, benzyloxycarbonyl-Asp(OCH₃)-Glu(OCH₃)-Val-Asp(OCH₃)—CH₂-fluoromethyl ketone and z-IETD-fmk.

Examples for pentapeptides as caspase inhibitors comprise for instance Z-VDVAD-fmk.

Abbreviations used for protecting groups above include: Z- (or z-), for benzyloxycarbonyl; BOC (or boc), for t-butyloxycarbonyl; Bzl, for benzyl; Fmoc, for 9-fluorenyloxycarbonyl; Ac, for acetyl; FMK (or fmk), for fluoromethyl ketone; CMK (or cmk), for chloromethyl ketone.

Furthermore, virus-encoded caspase inhibitors, such as the cowpox virus CrmA protein and the Bcl-2 oncoprotein or the caspase inhibitors Diap1, cIAP1, cIAP2, XIAP and p35, can also be used for the pharmaceutical composition and within the method for coating medical products.

The caspase inhibitors can be purchased from Enzyme Systems (Livermore Calif.).

The above mentioned caspase inhibitors can be used for the preparation of a pharmaceutical composition. Said pharmaceutical composition can furthermore be used for the coating of medical products such as artificial hearts, heart parts, lungs, arteries, veins, aortas, heart valves, corpse veins, valves, container, bags, cans, needles, catheter and parts especially artificial parts for the cardiovascular system and the extracorporeal circulation, surgical implants such as stents or catheters and devices for analytical purposes such as test tubes, titer plates, micro titer plates, well plates, analytical chips or material for chromatography such as gels, silica gels, columns, alumina, sepharose gels and the like. Most preferable are stents to be coated with a coating mixture such as the pharmaceutical composition mentioned above.

Preferred are caspase inhibitors consisting of two, three or four amino acids. Said di-, tri- or tetrapeptides can be used in their free form or with one or more protecting groups bond thereon.

As protecting groups, benzyloxycarbonyl, fluoromethyl ketone, chloromethylketone and t-butoxycarbonyl are most preferred.

One especially preferred caspase inhibitor is Ac-Tyr-Val-Ala-Asp-chloromethylketone (Ac-YVAD-CMK) as component of the coating of the above mentioned medical products.

It is also preferred that at least one amino acid of the above-mentioned caspase inhibitors has D-configuration, especially if one amino acid of Tyr-Val-Ala-Asp has D-configuration.

Neuropeptides as the proopiomelanocortin peptides (POMC), especially alpha-, beta- and gamma-melanocyte-stimulating hormone (MSH), more especially alpha-MSH, and Adrenocorticotropin (ACTH) and their related tripeptides (KPV), are known to have anti-inflammatory and immunosuppressive effects on the endothelial cells (Broad medical research program for the Eli and Edythe L. Broad Foundation; Kucharzik 2003). These properties reside in the C-terminal part of the tridecapeptide alpha-MSH and KPVs, which consists of three amino acids Lys-Pro-Val (Catania and Lipton, Endocrin. Rev. 1993, 14, 564-578; Bhardvaj et al., J. Immunol. 1996, 156, 2517-2521). MSH is structurally related to ACTH and is biologically generated from the precursor POMC. The two different species of MSH, α-MSH and β-MSH, have the first 13 amino acids in common with ACTH. Plasmalipotropin (LPH) and Cardiotropinlike peptide (CLIP) originate also from the precursor POMC and are presumed to have positive effects (Clin. Endocrin. & Metabol. 2001, 86(7); 2997-3000).

Thus, another aspect of the present invention relates to the use of compounds derived from the family of POMC-peptides is alpha-, beta- or gamma-MSH, ACTH, LPH or CLIP or protected, acylated, acetylated derivatives of said compounds for the coating of surfaces of medical product.

Some caspase inhibitors can be represented by the formula R-Lys-X. It was surprisingly found that not only caspase inhibitors but also oligopeptides and peptides of the general formula R-Lys-X are able to solve the problem underlying the invention. Said compounds including the caspase inhibitors which can also be used for the preparation of the pharmaceutical composition and for coating the surface of medical products are represented by the general formula R-Lys-X, wherein X represents a hydroxyl group, an amino group, a monoalkyl or dialkylamino group, an alkoxy group, an amino acid, an oligopeptide with 1-10 amino acids and wherein R is selected from the group comprising hydrogen, acyl group, acetyl group, an amino acid or a peptide with 2-70 amino acids.

Preferably, R represents a peptide having 3-50 amino acids, more preferably R represents a peptide having 5-35 amino acids, still more preferably R represents a peptide having 6-20 amino acids, further still more preferably R represents a peptide having 7-15 amino acids, still more preferably R represents a peptide having 8-12 amino acids, and most preferably R represents a peptide having 9-11 amino acids. Also most preferably R is a peptide of 10 amino acids.

Furthermore, it is advantageous when R represents a peptide comprising the tetrapeptide His-Phe-Arg-Trp or the tripeptides Phe-Arg-Trp or His-Phe-Arg.

Further preferred embodiments comprise compounds of general formula R-Lys-X wherein at least one amino acids of the residues R and/or X has D-configuration. More preferred are compounds wherein X comprises of L amino acids and R contains at least one D amino acid. Another more preferred embodiment of the present invention comprises compounds of general formula R-Lys-X wherein all amino acids of the residue X have L-configuration and all amino acids of the residue R have D-configuration. Within all mentioned embodiments it is also advantageous that the amino acid -Lys- in R-Lys-X has L-configuration.

According to the nomenclature of peptides, R is the residue leading to the N-terminal end of the peptide and X is the residue bound to the C-terminal end of the amino acid -Lys- in R-Lys-X.

Both ends, the C-terminal and the N-terminal end of the compound of general formula R-Lys-X, may be protected with common amino or carboxyl protecting groups such as acyl groups. Preferred amino protecting groups are acyl groups, such as formyl, acetyl, propionyl and preferably the acetyl group. Preferred protecting groups for carboxylic acids are monoalkylamino groups, dialkylamino groups, alkoxy groups, fluoromethyl ketones and chloromethyl ketones. Said protecting groups can be present at the C-terminal or N-terminal end or at both ends or at none of them.

A further preferred embodiment of the present invention comprises compounds of general formula R-Lys-X wherein X represents an oligopeptide selected from the group comprising Pharmaceutical composition according to any one of claims 5-18, wherein X represents an oligopeptide selected from the group comprising Pro, Pro-Thr, Pro-Val, Pro-Ala, Pro-Arg, Pro-Asn, Pro-Asp, Pro-Cys, Pro-Glu, Pro-Gin, Pro-Gly, Pro-His, Pro-Ile, Pro-Leu, Pro-Lys, Pro-Met, Pro-Phe, Pro-Pro, Pro-Ser, Pro-Trp, Pro-Thr-Thr, Pro-Thr-Val, Pro-Thr-Ala, Pro-Thr-Arg, Pro-Thr-Asn, Pro-Thr-Asp, Pro-Thr-Cys, Pro-Thr-Glu, Pro-Thr-Gln, Pro-Thr-Gly, Pro-Thr-His, Pro-Thr-Ile, Pro-Thr-Leu, Pro-Thr-Lys, Pro-Thr-Met, Pro-Thr-Phe, Pro-Thr-Pro, Pro-Thr-Ser, Pro-Thr-Trp, Pro-Val-Thr, Pro-Val-Val, Pro-Val-Ala, Pro-Val-Arg, Pro-Val-Asn, Pro-Val-Asp, Pro-Val-Cys, Pro-Val-Glu, Pro-Val-Gln, Pro-Val-Gly, Pro-Val-His, Pro-Val-Ile, Pro-Val-Leu, Pro-Val-Lys, Pro-Val-Met, Pro-Val-Phe, Pro-Val-Pro, Pro-Val-Ser, and Pro-Val-Trp.

Other preferred examples of compounds according to general formula R-Lys-X are selected from the group comprising R-Lys-Pro-X, R-Lys-Pro-Thr-X and R-Lys-Pro-Val-X.

Furthermore, the following compounds are preferred: R″-His-Phe-Arg-Trp-R′-Lys-X, R″-His-Phe-Arg-Trp-R′-Lys-Pro-X′, R″-His-Phe-Arg-Trp-R′-Lys-Pro-Thr-X′, R″-His-Phe-Arg-Trp-R′-Lys-Pro-Val-X′, R″-Phe-Arg-Trp-R′-Lys-X, R″-Phe-Arg-Trp-R′-Lys-Pro-X′, R″-Phe-Arg-Trp-R′-Lys-Pro-Thr-X′, R″-Phe-Arg-Trp-R′-Lys-Pro-Val-X′, R″-His-Phe-Arg-R′-Lys-X, R″-His-Phe-Arg-R′-Lys-Pro-X′, R″-His-Phe-Arg-R′-Lys-Pro-Thr-X′, and R″-His-Phe-Arg-R′-Lys-Pro-Val-X′

wherein X′ represents a hydroxyl group, an amino group, a monoalkyl or dialkylamino group, an alkoxy group, an amino acid, an oligopeptide with 1-8, preferably with 1-3 and more preferably with 1 or two amino acids and wherein R′ represents an oligopeptide of 1-10 amino acids and R″ is selected from the group comprising hydrogen, acyl group, acetyl group, an amino acid or a peptide with 1-0.60 amino-acids.

Most preferably, X′ represents one C-terminal protected or unprotected amino acid group. Furthermore, the L-configuration of X′ is preferred.

R′ is more preferable selected from the group comprising oligopeptide sequences of 1-5, still more preferably of 1-3 and most preferably of one or two amino acid residues. Furthermore, the L-configuration of the amino acids of R′ is preferred.

In addition thereto, N-terminal protected or unprotected peptides consisting of 1-40 amino acids, preferably 2-30, more preferably 3-20, still more preferably 3-13, still more preferably 4-7, and most preferably 5 or 6 are useful as residue R″. Furthermore, it is advantageous if at least one amino acid of the residue R″ has D-configuration. It is more advantageous if 10% and still more advantageous if 50% and most advantageous if more than 90% of the amino acids of R″ have D-configuration.

One especially preferred compound of general formula R-Lys-X is SYSMEHFRWGKPV. It is also preferred if one amino acid, more preferred if 3 amino acids, still more preferred if 6 amino acids and most preferred if more than 10 amino acids have D-configuration.

Also preferred are compounds of general formula R-Lys-X, wherein the compound of the formula R-Lys-X is derived from the family of POMC-peptides which have anti-inflammatory and antiimmunosuppressive properties.

Compounds of the formula Lys-X, wherein X represents a hydroxyl group, an amino group, an alkoxy group, Proline or Pro-Thr are known to have anti-inflammatory properties (WO 02/064131) and are suitable for coating compositions on medical devices. Derivatisations at the side chains of Lysine or Threonine are also possible without loosing the therapeutic character, chain extension up to the length of alpha-MSH and more offers a broad variety of derivatives.

Another aspect of the present invention relates to methods for coating medical products. Such methods comprise the steps of:
a) providing a surface of a medical product,
b) coating said surface with a coating composition comprising at least one caspase inhibitor and/or at least one compound of formula R-Lys-X.

The coating layer comprising the caspase inhibitor can be applied directly on the surface, normally an uncoated surface of the medical product. It is also possible to generate a first coating layer comprising of biologically stable and/or biodegradable polymers and to coat said first layer with a second layer comprising said caspase inhibitor and/or at least one compound of the general formula R-Lys-X, wherein R and X have the meanings as defined above. Said first coating layer may further comprise at least one anti-inflammatory, anti-prolific, anti-thrombotic, and/or anti-coagulative agent or said first coating layer may completely or mainly consist of said anti-inflammatory, anti-prolific, anti-thrombotic, and/or anti-coagulative agent. Preferably, the anti-inflammatory, anti-prolific, anti-thrombotic, and/or anti-coagulative agents as listed below are used with the coating methods.

Furthermore, it is advantageous to provide another layer as the outermost layer over or on top of the layer comprising said at least one caspase inhibitor and/or said at least one-compound of general formula R-Lys-X. The layer or the layers comprising a biologically stable polymer, a biodegradable polymer, at least one caspase inhibitor and/or at least one compound of general formula R-Lys-X may further comprise at least one anti-inflammatory, anti-prolific, anti-thrombotic, and/or anti-coagulative agent.

Preferably are coatings consisting of one or two layers. The layers, preferably the outermost layer can be designed in a way capable of allowing controlled release of the at least one anti-inflammatory, anti-prolific, anti-thrombotic, and/or anti-coagulative agent and/or the at least one caspase inhibitor and/or the at least one compound of general formula R-Lys-X.

It is also advantageous that the layer below or on top of the layer comprising the at least one compound of general formula R-Lys-X and/or the caspase inhibitor further comprises at least one anti-inflammatory, anti-prolific, anti-thrombotic, and/or anti-coagulative agent or that said layer completely or mainly consists of said anti-inflammatory, anti-prolific, anti-thrombotic, and/or anti-coagulative agent. Thus two embodiments are preferred: a) first layer consisting mainly or completely of at least one anti-inflammatory, anti-prolific, anti-thrombotic, and/or anti-coagulative agent, preferably Taxol® (paclitaxel), or a first layer consisting mainly of a biostable and/or biodegradable polymer, preferably selected from the group mentioned below, said layer comprising at least one anti-inflammatory, anti-prolific, anti-thrombotic, and/or anti-coagulative agent, preferably Taxol® (paclitaxel), and a second layer formed on said first layer containing said caspase inhibitor and/or said compound of general formula R-Lys-X or b) embodiments wherein the first and second layer is exchanged with each other Thus, it is possible to have one layer consisting of or mainly comprising said at least one anti-inflammatory, anti-prolific, anti-thrombotic, and/or anti-coagulative agent. It is also possible to have that at least one anti-inflammatory, anti-prolific, anti-thrombotic, and/or anti-coagulative agent incorporated into at least one layer comprising the biostabile and/or biodegradable polymer and/or the at least one compound of general formula R-Lys-X and/or the at least one caspase inhibitor. Furthermore, it is possible to have different anti-inflammatory, anti-prolific, anti-thrombotic, and/or anti-coagulative agents in different layers or to have the same anti-inflammatory, anti-prolific, anti-thrombotic, and/or anti-coagulative agent in different layers. Said agents and/or said compounds of general formula R-Lys-X and/or said caspase inhibitors can be released from different layers with different releasing rates or from the same layer with different releasing rates. The releasing rates are adjusted and controlled by the properties of the used polymer(s). Another preferred embodiment comprises a layer only consisting of at least one anti-inflammatory, anti-prolific, anti-thrombotic, and/or anti-coagulative agent and at least one compound of general formula R-Lys-X and/or at least one caspase inhibitor. Said embodiments preferably have one or two layers. The embodiments with two layers have one biostable and/or biodegradable polymer layer below or on top of said layer consisting only of at least one anti-inflammatory, anti-prolific, anti-thrombotic, and/or anti-coagulative agent and at least one compound of general formula R-Lys-X and/or at least one caspase inhibitor.

The term “biostable and biodegradable polymer” means either a composition of at least one biostable polymer and at least one biodegradable polymer or at least one block-polymer consisting of sequences which are biostabile and of sequences which are biodegradable.

The term “mainly” has the meaning of at least 85%, preferably at least 90%, more preferably more than 95%, still more preferably at least 98%, and most preferably more than 99%.

The layer containing said caspase inhibitor and/or said compound of general formula R-Lys-X and/or said anti-inflammatory, anti-prolific, anti-thrombotic, and/or anti-coagulative agent can be formed directly on the normally not hemocompatible surface of the medical product, or on a first layer applied on the surface of the medical product. On top of the layer containing said caspase inhibitor and/or said compound of general formula R-Lys-X and/or said anti-inflammatory, anti-prolific, anti-thrombotic, and/or anti-coagulative agent another layer can be generated.

Said outermost layer preferably comprises biologically stable and/or biodegradable polymers and more preferably consists mainly of biologically stable and/or biodegradable polymers. Moreover, said outermost layer may contain another anti-inflammatory, anti-prolific, anti-thrombotic, and/or anti-coagulative agent which may be identical or different from the agent used in a layer under said outermost layer. Another preferred embodiment contains an anti-inflammatory, anti-prolific, anti-thrombotic, and/or anti-coagulative agent only in the outermost layer.

The surface of the medical product may consist of metals, such as stainless steel or titan, alloys, ceramics, minerals, silicate materials such as glass, natural materials such as tissue, cells, biopolymers, synthetic polymers or plastics such as Teflon® (tetrafluoroethylene), PCV (polyvinyl chloride), polyethylene terephthalates, polyethylene, polypropylene, polyamides, polyurethanes, polycarbonates, polysulfones, polyether etherketones, silicones, polystyrene, polymethyl methacrylates, polyvinylidene fluorides and mixtures or copolymers of the aforementioned plastics and synthetic polymers.

As biostabile polymers may be used polyacrylic acid, polyacrylates, polymethylmethacrylates, polybutylmethacrylates, polyacrylamides, polyacrylonitriles, polyamides, polyether amides, polyethylene amines, polyimides, polycarbonates, polycarbourethanes, polyvinylketones, polyvinyl halides, polyvinylidene halides, polyvinyl ether, aromatic polyvinyls, polyvinyl esters, polyvinyl pyrollidones, polyoxymethylene, polyethylene, polypropylene, polytetrafluoroethylene, polyurethanes, polyolefin-elastomers, polyisobutylene, EPDM-gum, fluorosilicones, carboxymethyl chitosan, polyethylene terephtalat, polyvalerate, carboxymethyl celluloses, cellulose, rayon, rayontriacetate, cellulosenitrates, cellulose acetates, hydroxyethyl celluloses, cellulose butyrates, cellulose acetat-butyrates, ethylvinyl acetat-copolymeres, polysulfones, epoxy resins, ABS resins, EPDM-gum, silicones such as polysiloxanes, polyvinyl halides, and copolymeres, cellulose ether, cellulose triacetate, chitosan and copolymers and/or mixtures of the aforementioned polymers.

The biodegradable polymers can be selected from the group comprising polyvalerolactone, poly-ε-decalactone, polylactides, polyglycolides, copolymers of polylactide and polyglycolide, poly-ε-caprolacton, polyhydroxy butyric acid, polyhydroxy butyrate, polyhydroxy valerate, polyhydroxy butyrate-co-valerate, poly(1,4-dioxan-2,3-dione), poly(1,3-dioxan-2-one), poly-para-dioxanone, polyanhydrides such as polymaleic acid anhydride, polyhydroxy methacrylate, fibrin, polycyano acrylate, polycaprolacton dimethylacrylate, poly-β-maleic acid, polycaprolacton butyl-acrylate, multiblock polymers made of oligocaprolacton diole and oligodioxanon diole, polyether ester-multiblock polymers made of PEG and poly(butylenterephtalate, polypivotolactone, polyglycolic acid trimethyl-carbonate polycaprolacton-glycolide, poly(γ-ethylglutamate), poly(DTH-iminocarbonates), poly(DTE-co-DT-carbonates), poly(bisphenol A-iminocarbonates), polyorthoesters, polyglycolic acid trimethylcarbonates, polytrimethylcarbonates, polyiminocarbonates, poly(N-vinyl)-pyrrolidone, polyvinyl alcohols, polyester amides, glycolic polyester, polyphosphoesters, polyphosphazenes, poly[p-carboxyphenoxy)propane]polyhydroxypentanoic acid, polyanhydrides, polyethylenoxid-propylenoxid, smooth polyurethanes, polyurethanes bearing amino acid residues, polyether esters such as polyethylene oxid, polyalkenoxalates, polyorthoesters and copolymers thereof, carrageenanes, fibrinogen, starch, collagens, protein-based polymers, polyamino acids, synthetic polyamino acids, zein, modified zein, polyhydroxy alkanoates, pectinin acid, actinic acid, modified and unmodified fibrin and casein, carboxymethylsulfates, albumine, hyaluronic acid, heparan sulfates, heparin, chondroitin sulfates, dextranes, β-cyclodextrines, copolymere with PEG and/or polypropylen glycol, gum arabicum, guar, gelatine, collagens, collagen-N-hydroxysuccinimid, derivatives, modifications, copolymers and/or mixtures of the aforementioned biodegradable polymers.

The anti-inflammatory, anti-prolific, anti-thrombotic, and/or anti-coagulative agent may be selected from the group comprising:

Sirolimus (Rapamycin), Everolimus, Pimecrolimus, Somatostatin, Tacrolimus, Roxithromycin, Dunaimycin, Ascomycin, Bafilomycin, Erythromycin, Midecamycin, Josamycin, Concanamycin, Clarithromycin, Troleandomycin, Folimycin, Cerivastatin, Simvastatin, Lovastatin, Fluvastatin, Rosuvastatin, Atorvastatin, Pravastatin, Pitavastatin, Vinblastin, Vincristin, Vindesin, Vinorelbin, Etobosid, Teniposid, Nimustin, Carmustin, Lomustin, Cyclophosphamid, 4-hydroxy oxycyclophosphamide, Estramustin, Melphalan, Ifosfamid, Tropfosfamid, Chlorambucil, Bendamustin, Dacarbazin, Busulfan, Procarbazin, Treosulfan, Tremozolomid, Thiotepa, Daunorubicin, Doxorubicin, Aclarubicin, Epirubicin, Mitoxantron, Idarubicin, Bleomycin, Mitomycin, Dactinomycin, Methotrexat, Fludarabin, Fludarabin-5′-dihydrogenphosphat, Cladribin, Mercaptopurin, Thioguanin, Cytarabin, Fluorouracil, Gemcitabin, Capecitabiri, Docetaxel, Carboplatin, Cisplatin, Oxaliplatin, Amsacrin, Irinotecan, Topotecan, Hydroxycarbamid, Miltefosin, Pentostatin, Aldesleukin, Tretinoin, Asparaginase, Pegasparase, Anastrozol, Exemestan, Letrozol, Formestan, Aminoglutethemid, Adriamycin, Azithromycin, Spiramycin, Cepharantin, SMC-Proliferation-Inhibitor-2w, Epothilone A and B, Mitoxanthrone, Azathioprin, Mycophenolatmofetil, c-myc-Antisense, b-myc-Antisense, Betulinsaure, Camptothecin, PI-88 (sulfated oligosaccharide), Melanocyte-stimulating hormone (α-MSH), activated protein C, IL1-β-inhibitor, Thymosin α-1, fumaric acids and esters thereof, Calcipotriol, Tacalcitol, Lapachol, β-Lapachon, Podophyllotoxin, Betulin, podophyllic acids 2-ethylhydrazide, Molgramostim (rhuGM-CSF), Peginterferon α-2b, Lanograstim (r-HuG-CSF), Filgrastim, Macrogol, Dacarbazin, Basiliximab, Daclizumab, Selectin (Cytokin antagonist), CETP-Inhibitor, Cadherine, Cytokininhibitoren, COX-2-inhibitor, NFkB, Angiopeptin, Ciprofloxacin, Camptothecin, Fluoroblastin, monoclonal antibodies which inhibit proliferation of muscle cells, bFGF-antagonists, Probucol, Prostaglandine, 1,11-dimethoxycanthin-6-one, 1-hydroxy-11-methoxycanthin-6-one, Scopolectin, Colchicin, NO donors such as pentaerythrityltetranitrate and Syndnoeimine, S-nitroso derivatives, Tamoxifen, Staurosporin, β-Estradiol; α-Estradiol, Estriol, Estron, Ethinylestradiol, Fosfestrol, Medroxyprogesteron, Estradiolcypionate, Estradiolbenzoate, Tranilast, Kamebakaurin and other terpenoides which are used in cancer therapy, Verapamil, Tyrosin-Kinase-inhibitors (Tyrphostine), Cyclosporin A, Paclitaxel and derivatives thereof such as 6-α-hydroxy-Paclitaxel, Baccatin, Taxotere, synthetic macrocyclic oligomers of carbonsuboxids (MCS) and derivatives thereof, Mofebutazon, Acemetacin, Diclofenac, Lonazolac, Dapson, o-Carbamoylphenoxy acetic acid, Lidocain, Ketoprofen, Mefenaminsäure, Piroxicam, Meloxicam, chloroquinphosphate, Penicillamin, Tumstatin, Avastin, D-24851, SC-58125, hydroxychloroquin, Auranofin, Natriumaurothiomalat, Oxaceprol, Celecoxib, β-Sitosterin, Ademetionin, Myrtecain, Polidocanol, Nonivamid, Levomenthol, Benzocain, Aescin, Ellipticin, D-24851 (Calbiochem), Colcemid, Cytochalasin A-E, Indanocine, Nocadazole, S 100 protein, Bacitracin, Vitronectin-receptor antagonists, Azelastin, Guanidylcyclase-stimulator, inhibitors of metallproteinase-1 and 2, free nucleic acids, nucleic acids incorporated into virus hosts, DNA- and RNA-fragments, Plaminogen-activator inhibitor-1, Plasminogen-activator inhibitor-2, Antisense oligonucleotides, VEGF-inhibitors, IGF-1, antibiotics such as Cefadroxil, Cefazolin, Cefaclor, Cefotixin, Tobramycin, Gentamycin, Penicillines such as Dicloxacillin, Oxacillin, Sulfonamide, Metronidazol, antithrombotics such as Argatroban, Aspirin, Abciximab, synthetic Antithrombin, Bivalirudin, Coumadin, Enoxoparin, desulfated and N-reacetylated heparin, Tissue-Plasminogen-activator, GpIIb/IIIa-platelet membrane receptor, factor Xₐ-inhibitor antibody, Heparin, Hirudin, r-Hirudin, PPACK, Protamin, sodium salt of 2-methylthiazolidin-2,4-dicarboxylic acid (Thialin-Na), Prourokinase, Streptokinase, Warfarin, Urokinase, Vasodilatoren such as Dipyramidol, Trapidil, Nitroprusside, PDGF-antagonists such as Triazolopyrimidin and Seramin, ACE-inhibitors such as Captopril, Cilazapril, Lisinopril, Enalapril, Losartan, Thioproteaseinhibitoren, Prostacyclin, Vapiprost, Interferon α, β and γ, Histamin antagonists, Serotoninblocker, apoptosis inhibitors, apoptosis regulators such as p65, NF-kB or Bcl-xL-antisense-oligonucleotids, Halofuginon, Nifedipin, Tocopherol, Vitamin B1, B2, B6 and B12, folic acid, Tranirast, Molsidomin, Teepolyphenole, Epicatechingallat, Epigallocatechingallat, Boswellinic acid and its derivatives, Leflunomid, Anakinra, Etanercept, Sulfasalazin, Etoposid, Dicloxacyllin, Tetracyclin, Triamcinolon, Mutamycin, Procainimid, D24851, SC-58125, retinoic acid, Quinidin, Disopyrimid, Flecainid, Propafenon, Sotolol, Amidoron, natural and synthetic steroides such as Bryophyllin A, Inotodiol, Maquirosid A, Ghalakinosid, Mansonin, Streblosid, Hydrocortison, Betamethason, Dexamethason, none-steroidal substances (NSAIDS) such as Fenoporfen, Ibuprofen, Indomethacin, Naproxen, Phenylbutazon and other antiviral agents such as Acyclovir, Ganciclovir and Zidovudin, antimycotics such Clotrimazol, Flucytosin, Griseofulvin, Ketoconazol, Miconazol, Nystatin, Terbinafin, antiprozoal agents such as Chloroquin, Mefloquin, Quinin, natural Terpenoides such as Hippocaesculin, Barringtogenol-C21-angelat, 14-Dehydroagrostistachin, Agroskerin, Agroskerin, Agrostistachin, 17-Hydroxyagrostistachin, Ovatodiolide, 4,7-oxycycloanisomelic acid, Baccharinoide B1, B2, B3 and B7, Tubeimosid, Bruceanole A, B and C, Bruceantinoside C, Yadanzioside N and P, Isodeoxyelephantopin, Tomenphantopin A and B, Coronarin A, B, C and D, Ursolic acid, Hyptatic acid A, Zeorin, Iso-Iridogermanal; Maytenfoliol, Effusantin A, Excisanin A and B, Longikaurin B, Sculponeatin C, Kamebaunin, Leukamenin A and B, 13,18-dehydro-6-alpha-Senecioyloxychaparrin, Taxamairin A and B, Regenilol, Triptolid, Cyrnarin, Apocymarin, Aristolochic acid, Anopterin, Hydroxyanopterin, Anemonin, Protoanemonin, Berberin, Cheliburinchloride, Cictoxin, Sinococulin, Bombrestatin A and B, Cudraisoflavon A, Curcumin, Dihydronitidin, Nitidinchloride, 12-beta-hydroxypregnadien 3,20-dione, Bilobol, Ginkgol, Ginkgolsäure, Helenalin, Indicin, Indicin-N-oxide, Lasiocarpin, Inotodiol, glycoside la, Podophyllotoxin, Justicidin A and B, Larreatin, Malloterin, Mallotochromanol, Isobutyrylmallotochromanol, Maquirosid A, Marchantin A, Maytansin, Lycoridicin, Margetin, Pancratistatin, Liriodenin, Oxoushinsunin, Aristolactam-AII, Bisparthenolidin, Periplocosid A, Ghalakinoside, Ursolic acid, deQxypsorospermin, Psycorubin, Ricin A, Sanguinarin, Manwuweioic acid, methylsorbifolin, Sphatheliachromen, Stizophyllin, Mansonin, Streblosid, Akagerin, Dihydrousambaraensin, Hydroxyusambarin, Strychnopentamin, Strychnophyllin, Usambarin, Usambarensin, Berberin, Liriodenin, Oxoushinsunin, Daphnoretin, Lariciresinol, Methoxylariciresinol, Syringaresinol, Umbelliferon, Afromoson, Acetylvismion B, Desacetylvismion A, Vismion A and B and sulfur containing amino acids such as cystin and salts thereof and/or mixtures of the above mentioned agents. Another aspect of the present invention relates to medical products obtainable according to one of the coating methods described above. Most preferably, the coated medical products are stents.

Preferred anti-inflammatory, anti-prolific, anti-thrombotic, and/or anti-coagulative agents are: Tacrolimus, Pimecrolimus, PI 88, Paclitaxel and derivatives thereof, Trapidil, α- and β-Estradiol, 2-methylthiazolidin-2,4-dicarboxylic acid and salts thereof, preferably sodium salts, macrocyclic carbon suboxyd (MCS) and derivatives thereof, Sirolimus, fumaric acid and esters thereof, activated protein C, interleucin 11-inhibitors and melanocyte-stimulating hormone (α-MSH), cystin, Ellipticin, Bohemin, Indanocin, Colcemid and derivatives thereof, methionin and salts thereof and/or mixtures of the aforementioned agents.

Taxol® (paclitaxel) is the most preferred anti-inflammatory, anti-prolific, anti-thrombotic, and/or anti-coagulative agent.

### EXAMPLES

The way of action of stents coated according to the present invention was investigated using animal models.

An increased amount of apoptotic smooth muscle cells in coronary arteries of pigs could be detected 30 minutes after a balloon angioplasty. Thereafter, the adventitia and the neointima were separately analyzed and different time-depending changes in the rate of apoptosis were measured. The highest levels of apoptotic smooth muscle cells, inflammatory cells, and fibroblast cells of the adventitia respectively were detected 18 hours, 6 hours and 7 days after PTCA (percutaneous transluminal coronary angioplasty). A quantitative determination of the rate of apoptosis in the different cell types and vessel wall layers after balloon angioplasty and stent implantation was conducted as follows:

Domestic pigs having a weight between 20 and 30 kg were fed with normal feed without the addition of fat supplementaries during the whole test. The pigs were kept fasting over night and were thereafter sedated using 30 mg/kg body weight of ketamine, 12 mg azepromazin and 1 ampoule of rubinol. 5 mg/kg of thiopental were administered before intubation. The pigs were given an artificial respiration by use of a mixture of 20% pure oxygen and 80% normal air after endotracheal intubation. Endotracheal intubation is a procedure by which a tube is inserted through the mouth down into the trachea (the large airway from the mouth to the lungs). After administration of 0.1 mg fentanyl and 2.5 mg aceproazin into the bolus, the anaesthesia was maintained by administration of 0.08 mg/kg fentanyl (0.05 mg/ml infusion). Procain-Penicillin G (200,000 IU/ml) and dihydro stretomycinsulfate (200 mg/10 kg body weight) were administered intramuscularly for the purpose of antibiotic protection.

Thereafter, an arteriotomy of the A. carotis communis dextra was carried out under sterile conditions and a 7F-channel was introduced. Puls, arterial blood pressure, and body temperature were measured during the whole operation. Additionally, the blood gases and the acid-base-metabolism were controlled in samples of arterial blood. After the administration of 200 IU/kg body weight of heparin and 250 mg/kg acetylsalicylic acid, a 7F catheter was inserted into the aorta ascendens. Additional 400 IU of heparin per hour were administered via infusion. The angiography of the right and left coronary artery was carried out by the use of non-ionic contrast agents after intracoronary administration of 200 μg nitroglycerin.

One artery of the left vascular system (either A. interventricularis or A. circumflexa) was randomly selected for stent implantation and the other artery was used for balloon angioplasty. The arteria coronaria was used as untreated control vessel.

A balloon having at least a balloon-vessel-ratio of 1.3:1 was used for balloon angioplasty in order to hurt the artery by overexpansion. The vessel was dilated (expanded) three times at the same position for 30 seconds and a pressure of 6 atm (atmospheres).

Thereafter, stents having a length of 15 mm were implanted according to standard methods. The diameter of the stent was selected in the way that a stent-vessel-ratio of 1.1:1 was obtained. During implantation, the stent balloon was blown up three times for 30 seconds applying a pressure of 6 bar.

An angiography of the right coronary artery was performed after a control angiography of the treated vessels had been carried out. Then, the catheter and the channel were removed and after ligation of the place of arteriotomy, the fascia and the skin were sewed up. Thereafter, the anaesthesia was stopped and the antibiotics trimethoprim and sulfadoxin together with the analgesic drug metamizol were administered. In addition thereto, 250 mg acetylsalicylic acid was given per day and per os (oral) after the intervention during the remaining live time of the animals in order to prevent acute or subacute thrombosis caused by the stent.

After 4 weeks a control angiography of the right and left coronary system was performed and an intravascular ultrasonic examination of the stent and the place treated with the dilated balloon was conducted.

Thereafter the pigs were euthanized by intravenous injection of 10 ml of a saturated potassium chloride solution. The hearts of the pigs were retained and washed with a sodium chloride solution. Thereafter, a pressure fixation was performed by use of buffered formaldehyde (4%) and about 100-110 mmHG perfusion pressure. Then, the coronary arteries of the heart were cut off, stored for 24 hours in buffered formaldehyde (2%) and thereafter in paraffin. The stent was removed using a microscope before storing the vessel in paraffin in order to prevent injury of the vessel.

The caspase inhibitor Ac-Tyr-Val-Ala-Asp-chloromethylketone (Ac-YVAD-CMK) was locally administered during the test period via a perfusion balloon by means of a poly-lumen catheter. Said catheter consists of an infusion connector, a catheter body and distal infusion regions comprising 4 separate lumens.

One group of test animals received the caspase inhibitor while another group were kept untreated as control group. The neointimal proliferation was macroscopically assessed via IVUS whereby the analyses were carried out 4 weeks after balloon angioplasty and stent implantation. All IVUS measurements were evaluated off-line by means of a computer-based IVUS analysis system. The qualitative IVUS analysis comprises an assessment of the plaque composition (hard or smooth, thrombus, tear plaque or calcification respectively) and the eccentricity. The neointimal proliferation was calculated as average of 3 values. Moreover, histological investigations were performed. For this purpose, cuts of each segment of the artery were colored with hematoxylin-eosin and Verhoeff-van-Gieson in order to indicate injuries of the vessel caused by the intervention.

Quantitative evaluation of the injuries of the vessel and the neointimal response to the stent implantation was performed using the cuts colored according to Verhoeff-van-Gieson by applying a method created by Schwartz et al. Each wire of the stent was assessed and classified according to the severity of the injuries caused by this wire and the position of said wire in the histological layers of the vascular wall.

For the identification of macrophages and smooth muscle cells, anti-rabbit-macrophages-antibodies of mice (RAM 11, DAKO Corp.) and anti-rabbit-smooth-muscle-cells-alpha-actin-monoclonal-antibodies of mice were used. Proliferating cells were detected by marking the cuts with mouse antibodies-against PCNA (proliferating cell nuclear antigen; clone PC 10, DAKO). For this purpose, the tissue was incubated for 1 hour together with primary antibodies at 37° C. in a humidified chamber. The binding of the antibodies was achieved applying an indirect biotin-streptavidin horseradish peroxidase (Amersham) or alkaline phosphatase (Sigma) method. The methods were carried out according to the instructions of the supplier.

Finally, an in situ evaluation of apoptotic cells was performed. For this purpose the terminal transferase-mediated dUTP nick end labeling kit (TUNEL), a kit for displaying apoptosis in situ, was used according to the supplier's instructions. Thereby, positive controls were treated with DNAse after fixation and permeabilization in order to cleave DNA and to obtain DNA strand pieces. Simultaneously, negative controls were stained with a staining solution (without terminal transferase) instead of the TUNEL reaction mixture. The binding of antibodies were visualized with diaminobenzidine (Pierce). The reaction with diaminobenzidine causes a brown color.

It could be demonstrated that said parts of the blood vessel which were treated with an apoptosis inhibitor (in the present case with Ac-YVAD-CMK) showed a reduction of plaque volume to approximately ⅙, a reduction of maximum plaque area to approximately ⅓ and a reduction of the stenotisized (the area which comes into contact with the introduced stent) area to approximately 40% in comparison with the values obtained from the negative control group. 7 pigs were used for each group, the positive and the negative control group.

The publications cited above are incorporated herein by reference.

From the foregoing description, additional embodiments of the present invention will be immediately apparent to those skilled in the art. All such embodiments are intended to be encompassed by the invention disclosed herein and as defined in the claims to follow.

We claim:

1. A pharmaceutical composition comprising a caspase inhibitor and/or a compound of the formula R-Lys-X and at least one pharmaceutically acceptable carrier, polymer matrix, solvent and/or diluents.

2. The pharmaceutical composition according to claim 1, wherein the caspase inhibitor is selected from the group consisting of benzyloxycarbonyl-Val-Ala-Asp-fluoromethyl ketone, Ile-Glu-Thr-Asp-fluoromethyl ketone, t-butoxycarbonyl-Asp(OCH₃)—CH₂F, boc-aspartyl(OMe)-fluoromethylketone (BAF) and BOC-Asp-FMK (BD), BD-fmk, Z-FA-fmk, z-VAD, z-Val-Ala-Asp-fluoromethylketone (z-VAD-fmk), IAP, benzyloxycarbonyl-Val-Ala-Asp(OCH₃)—CH₂-fluoromethyl ketone, benzyloxycarbonyl-Ile-Glu(OCH₃)-Thr-Asp(OCH₃)—CH₂-fluoromethyl ketone, Z-AAD-fmk, DEVD, Ac-DEVD-CHO, Z-Asp-CH₂-DCB, acetyl-Asp-Glu-Val-Asp-fluoromethyl-ketone (Ac-DEVD-FMK), YVAD, acetyl-Tyr-Val-Ala-Asp-chloromethyl-ketone (Ac-YVAD-CMK), z-DEVD-fmk, benzyloxycarbonyl-Asp(OCH₃)-Glu(OCH₃)-Val-Asp(OCH₃)—CH₂-fluoromethyl ketone, z-IETD-fmk, Z-VDVAD-fmk, CrmA, Bcl-2, Diap1, cIAP1, cIAP2, XIAP and p35.

3. The pharmaceutical composition according to claim 1, wherein the caspase inhibitor is a di, tri, tetra or pentapeptide covalently bound to chloromethylketone.

4. The pharmaceutical composition according to claim 3, wherein the caspase inhibitor is Ac-Tyr-Val-Ala-Asp-chloromethylketone (Ac-YVAD-CMK).

5. The pharmaceutical composition according to claim 1, wherein the compound of the formula R-Lys-X is selected from the group wherein X represents a hydroxyl group, an amino group, a monoalkyl or dialkylamino group, an alkoxy group, an amino acid, an oligopeptide with 1-10 amino acids and wherein R is selected from the group comprising hydrogen, acyl group, acetyl group, an amino acid or a peptide with 2-70 amino acids.

6. The pharmaceutical composition according to claim 5, wherein R represents a peptide having 8-12 amino acids.

7. The pharmaceutical composition according to claim 5, wherein R represents a peptide comprising the tetrapeptide His-Phe-Arg-Trp.

8. The pharmaceutical composition according to claim 5, wherein R represents a peptide comprising the tripeptide Phe-Arg-Trp.

9. The pharmaceutical composition according to claim 5, wherein R represents a peptide comprising the tripeptide His-Phe-Arg.

10. The pharmaceutical composition according to claim 5, wherein R represents a peptide comprising at least one amino acid having D configuration.

11. The pharmaceutical composition according to claim 5, wherein R represents a peptide consisting of amino acids having D configuration.

12. The pharmaceutical composition according to claim 5, wherein R represents a peptide bearing an acyl group or acetyl group at the N-terminal end.

13. The pharmaceutical composition according to claim 5, Wherein X represents an oligopeptide selected from the group comprising Pro, Pro-Thr, Pro-Val, Pro-Ala, Pro-Arg, Pro-Asn, Pro-Asp, Pro-Cys, Pro-Glu, Pro-Gln, Pro-Gly, Pro-His, Pro-Ile, Pro-Leu, Pro-Lys, Pro-Met, Pro-Phe, Pro-Pro, Pro-Ser, Pro-Trp, Pro-Thr-Thr, Pro-Thr-Val, Pro-Thr-Ala, Pro-Thr-Arg, Pro-Thr-Asn, Pro-Thr-Asp, Pro-Thr-Cys, Pro-Thr-Glu, Pro-Thr-Gln, Pro-Thr-Gly, Pro-Thr-His, Pro-Thr-Ile, Pro-Thr-Leu, Pro-Thr-Lys, Pro-Thr-Met, Pro-Thr-Phe, Pro-Thr-Pro, Pro-Thr-Ser, Pro-Thr-Trp, Pro-Val-Thr, Pro-Val-Val, Pro-Val-Ala, Pro-Val-Arg, Pro-Val-Asn, Pro-Val-Asp, Pro-Val-Cys, Pro-Val-Glu, Pro-Val-Gln, Pro-Val-Gly, Pro-Val-His, Pro-Val-Ile, Pro-Val-Leu, Pro-Val-Lys, Pro-Val-Met, Pro-Val-Phe, Pro-Val-Pro, Pro-Val-Ser, and Pro-Val-Trp.

14. The pharmaceutical composition according to claim 13, wherein X represents an oligopeptide selected from the group comprising Pro, Pro-Thr or Pro-Val.

15. The pharmaceutical composition according to claim 5, wherein X represents an oligopeptide bearing an amino group, a monoalkyl or dialkylamino group, an alkoxy group, a fluoromethyl ketone or a chloromethyl-ketone at the C-terminal end.

16. The pharmaceutical composition according to claim 5, wherein X represents an oligopeptide comprising at least one amino acid having D configuration.

17. The pharmaceutical composition according to claim 5, wherein the compound of formula R-Lys-X is SYSMEHFRWGKPV.

18. The pharmaceutical composition according to claim 17, wherein at least one amino acid of the compound SYSMEHFRWGKPV has D-configuration.

19. The pharmaceutical composition according to claim 5, wherein the compound of general formula R-Lys-X is a compound derived from the family of POMC-peptides which have anti-inflammatory and antiimmunosuppresive properties.

20. The pharmaceutical composition according to claim 19, wherein the compound derived from the family of POMC-peptides is alpha-, beta- or gamma-MSH, ACTH, LPH or CLIP or protected, acylated, acetylated derivatives of said compounds.

21. The pharmaceutical composition according to claim 5, further comprising at least one anti-inflammatory, anti-prolific, anti-thrombotic, and/or anti-coagulative agent.

22. A method for the preparation of a hemocompatibly coated medical product, comprising the steps of:
a) providing a surface of a medical product,
b) coating said surface with a coating composition comprising at least one caspase inhibitor and/or at least one compound of formula R-Lys-X.

23. The method for the preparation of a hemocompatibly coated medical product according to claim 22, further comprising the step c);
c) coating said caspase inhibitor and/or compound of general formula R-Lys-X containing layer with a layer comprising biologically stable and/or biodegradable polymers.

24. A method for the preparation of a hemocompatibly coated medical product, comprising the steps of:
a) providing a surface of a medical product,
b′) coating said surface with a first layer comprising biologically stable and/or biodegradable polymers and
b″) coating said first layer with a coating composition comprising at least one caspase inhibitor and/or at least one compound of formula R-Lys-X.

25. The method for the preparation of a hemocompatibly coated medical product according to claim 24, further comprising the step c):
c) coating said caspase inhibitor and/or compound of general formula R-Lys-X containing layer with a layer comprising biologically stable and/or biodegradable polymers.

26. The method according to any one of claims 22-25, wherein the layer of biologically stable and/or biodegradable polymers and/or the layer containing said caspase inhibitor and/or said compound of general formula R-Lys-X further comprises an anti-inflammatory, anti-prolific, anti-thrombotic, and/or anti-coagulative agent.

27. The method according to any one of claims 22-25, wherein the caspase inhibitor is selected from the group consisting of benzyloxycarbonyl-Val-Ala-Asp-fluoromethyl ketone, Ile-Glu-Thr-Asp-fluoromethyl ketone, t-butoxycarbonyl-Asp(OCH₃)—CH₂F, boc-aspartyl(OMe)-fluoromethylketone (BAF) and BOC-Asp-FMK (BD), BD-fmk, Z-FA-fmk, z-VAD, z-Val-Ala-Asp-fluoromethylketone (z-VAD-fmk), IAP, benzyloxycarbonyl-Val-Ala-Asp(OCH₃)—CH₂-fluoromethyl ketone, benzyloxycarbonyl-Ile-Glu(OCH₃)-Thr-Asp(OCH₃)—CH₂-fluoromethyl ketone, Z-AAD-fmk, DEVD, Ac-DEVD-CHO, Z-Asp-CH₂-DCB, acetyl-Asp-Glu-Val-Asp-fluoromethyl-ketone (Ac-DEVD-FMK), YVAD, acetyl-Tyr-Val-Ala-Asp-chloromethyl-ketone (Ac-YVAD-CMK), z-DEVD-fmk, benzyloxycarbonyl-Asp(OCH₃)-Glu(OCH₃)-Val-Asp(OCH₃)—CH₂-fluoromethyl ketone, z-IETD-fmk, Z-VDVAD-fmk, CrmA, Bcl-2, Diap1, cIAP1, cIAP2, XIAP and p35.

28. Method according to any one of claims 22-25, wherein the compound of general formula R-Lys-X is selected from the group wherein X represents a hydroxyl group, an amino group, a monoalkyl or dialkylamino group, an alkoxy group, an amino acid, an oligopeptide with 1-10 amino acids and wherein R is selected from the group comprising hydrogen, acyl group, acetyl group, an amino acid or a peptide with 2-70 amino acids.

29. A coated medical product obtained according to the method of any one of claims 22-25.

30. The medical product according to claim 29, wherein the medical product is a stent.
